(19) United States
(12) Patent Application Publication
(10) Pub. No.: US 2008/0286830 A1
(43) Pub. Date: Nov. 20, 2008

(54) Analysing Breath Samples for Volatile Organic Compound

(76) Inventors: Jennifer M. Scotter, Christchurch (NZ); Stephen T. Chambers, Christchurch (NZ); Mona Syhre, Christchurch (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 11/911,925

(22) PCT Filed: Apr. 13, 2006
(86) PCT No.: PCT/NZ2006/000071

§ 371 Date: Jun. 24, 2008

(30) Foreign Application Priority Data
Apr. 19, 2005 (NZ) 539518

(51) Int. Cl. C12Q 1/04

(52) U.S. Cl. 435/34

(57) ABSTRACT

The invention describes the use of a biomarker pentylfuran to detect bacterial and/or fungal pathogens. Pentylfuran is released from certain pathogens and detected in the headspace gas of an in vitro culture or in the breath sample of a patient. Pentylfuran is particularly useful in the detection of Aspergillus species, especially Aspergillus fumigatus which is a pathogen of humans.

### FIELD OF THE INVENTION

The invention relates to the determination of a unique biomarker pentylfuran and to its use to test for fungal and bacterial pathogens, including Aspergillus fumigatus in an individual or from a culture using analysis of breath/air samples.

### BACKGROUND TO THE INVENTION

All micro-organisms produce by-products as a result of their normal metabolism. The ability of different organisms to metabolise different substrates in order to satisfy their energy and nutritional requirements is fundamental to laboratory microbiology, and forms the basis of many rapid-identification tests. The metabolites produced by a single species can vary widely, depending upon the growth substrate, conditions (temperature, oxygen availability), and the age of the culture itself.

Amongst the many large primary and secondary metabolites produced by microbes, some organic substances are formed which readily volatilise at low temperatures. Microbial volatile organic compounds (MVOCs) have been studied extensively in agriculture and food production, as some MVOCs have important health and economic implications in these fields. For example, some MVOCs have been associated with spoilage in stored crops and foodstuffs, where they may be responsible for tainted, “off” flavours, discolouring of products, or toxicity. Profiles of MVOCs are increasingly being found to be unique to the species or strain level.

Invasive aspergillosis is one of the most problematic infections due to difficulties of diagnosis and treatment. Volatile organic compounds (VOCs) have the potential to improve the specificity and sensitivity of diagnosis of this and other infections. It would be useful to identify a unique biomarker of Aspergillus species, particularly Aspergillus fumigatus, in the headspace gas of in vitro cultures and to detect the marker from breath samples of infected or colonised patients.

### OBJECT OF THE INVENTION

It is an object of the invention to provide a biomarker to detect bacterial and/or fungal pathogens such as Aspergillus fumigatus in a biosample or at least to provide the public with a useful choice.

### SUMMARY OF THE INVENTION

The invention provides the biomarker, pentylfuran for the use in bio analysis of microorganisms such as fungal and bacterial pathogens. In particular, the invention provides the use of the biomarker pentylfuran to detect fungal species, more particularly, Aspergillus species in a biosample.

More particularly the invention provides the use of the biomarker pentylfuran to detect Aspergillus fumigatus in a biosample.

The biosample is preferably a gaseous biological source.

The biosample may, most preferably, be the headspace gas of an in vitro culture or a breath sample of a patient, or another biosample such as a sputum sample.

In particular, the invention provides the use of the biomarker pentylfuran, in the bio analysis of microorganisms in a gaseous bio sample from breath of an animal, including a human.

Other micro organisms such as Aspergillus flavus, Haemophilus influenza and Pseudomonas aeruginosa may also be detected. The biosample is preferably a breath sample. Preferably the biomarker is 2-pentylfuran. However it could be 3-pentylfuran.

The invention provides a method of detecting Aspergillus in a patient comprising:
(a) obtaining a biosample from the breath of the patient;
(b) analysing the biosample for the presence of pentylfuran; and
(c) determining whether Aspergillus is present in the breath sample.

The invention also provides a method of detecting Aspergillus species in an in vitro culture comprising:
a. obtaining a biosample from the headspace gas of the in vitro culture;
b. analysing the biosample for the presence of pentylfuran; and
c. determining whether Aspergillus is present in the in vitro culture.

The invention also provides the use of a biomarker pentylfuran in the detection of Aspergillus fumigatus from a breath sample of an animal or from an air sample from a culture

The Aspergillus species is preferably Aspergillus fumigatus.

### DESCRIPTION OF THE DRAWINGS

FIG. 1 shows a chest CT scan of a patient with multiple foci of air space opacity surrounded by hyperdense material.

### DETAILED DESCRIPTION OF THE INVENTION

The invention will now be described, by way of example only.

Gas Chromatography-Mass Spectroscopy (GC-MS) combined with Solid Phase Micro Extraction (SPME) was used to identify pentylfuran as a specific biomarker of A. fumigatus from cultures. Four litre breath samples were collected from patients with Cystic Fibrosis, with or without colonisation of A. fumigatus and other pathogens, and healthy volunteers. Breath samples were semi-quantitatively analysed by SPME/GC-MS for presence or absence of pentylfuran.

A total of 21 individuals were tested. Pentylfuran was detected from breath samples of 4/4 patients with CF and A. fumigatus colonisation, 3/7 patients with CF and no microbiological evidence of A. fumigatus and 0/10 healthy control individuals.

### Materials and Methods

### Strains and Culture Conditions

Clinical isolates of Aspergillus flavus, Aspergillus fumigatus, Candida albicans, Mucor racemosus, Fusarium solani, and Cryptococcus neoformans were used in these experiments.

Organisms were grown on blood agar within 100 ml sterile glass vials stoppered with airtight aluminium caps incorporating a teflon-coated rubber septum.

### Strain Preparation.

Strains were grown for 72 hours on blood agar plates, then, in the case of yeast species, a sterile loop of culture was removed from the plate and transferred to 5 mL of sterile water. For filamentous fungal strains, spores were harvested from the plate with sterile water containing 0.05% Tween. Five hundred microlitres of this suspension was introduced to the sealed culture vial by injecting through the septum onto the medium.

Cultures were maintained at 37° C. for 5 days. Vials were flushed with 100 ml purified dry air once every approximately 12 hours.

### Detection of Pentylfuran by GC-MS, Calibration and Standardisation

Calibration curves of headspace gas analysis of serial aqueous dilutions of pentylfuran were plotted. The resulting calibration curve proved to be linear in the range 1-50 pg.

### Patient Demographics

Four participants with cystic fibrosis and Aspergillus colonisation, 6 patients with CF and no Aspergillus colonisation, and 10 control individuals meeting the inclusion criteria for the study were identified. Relevant demographic and clinical data are shown in table 2.

### Solid-Phase Micro Extraction (SPME Gas Chromatography-Mass Spectrometry (GC-MS).

A comprehensive literature review was performed, and a database of all reported MVOCs from Aspergillus species was created, which included the compound isolated, species and strain, culture medium and conditions, and analysis method. Headspace gases of strains cultured as described above were subjected to SPME/GC-MS analysis.

### Sample Preparation

The conditioned SPME fibre was exposed into culture vials for 10 minutes and then desorbed directly in the injection port for 5 minutes.

### GC/MS Parameters

The temperatures of the injector, ion trap, manifold and transfer line were 250, 200, 60 and 250° C. respectively. The oven program commenced at 50° C. for 2 minutes and was raised to 250° C. at a rate of 10° C./min. at which temperature it was maintained for a further 2 minutes. Helium flow was set at a constant rate of 1.2 mL/min. The split vent was opened to a ratio of 1:50 after 1 minute. Fragmentation was performed in the EI-mode as full scan which gave additional certainty. Further MS/MS fragmentation could be used to further increase sensitivity.

### Calibration and Semi-Quantification

Fifty microlitres of diluted pentylfuran solutions in methanol were deposited into 20 mL headspace vials. The final calibration was made up as total amounts in the headspace vials containing 1, 5, 10 and 50 pg, respectively. The fibre was exposed into the headspace vials for 5 min using the Combi-PAL autosampler.

### Patient Selection

Patients enrolled on the study included individuals with cystic fibrosis (CF) colonised chronically with aspergillus, patients with CF not colonised with aspergillus, and healthy control individuals. Ethical approval for the study was obtained from the local ethics committee, and participants gave their informed consent to take part in the study. Inclusion criteria for patients to act as “positives” (colonised with Aspergillus) were a history of positive culture for A. fumigatus from lower respiratory tract specimens (sputum, BAL, tracheal aspirate, or cough swab in cases where sputum had not been made available to the microbiology laboratory). Patients needed to have a minimum of three positive results within the past 12 months, and ideally one from within 1 month of testing. Patients were excluded from the study if they were currently undergoing treatment with itraconazole for ABPA.

Patients in the cystic fibrosis “uncolonised” group were selected if they had no current or past history of positive culture results for Aspergillus as described above, and no clinical evidence of aspergillus colonisation. Healthy control individuals were recruited from laboratory staff. These participants were asked to complete a questionnaire which served to give information concerning any recent antibiotic use or evidence of respiratory or urinary tract infection. Any participants not meeting these criteria were excluded from the study.

### Breath Sampling

Breath samples were collected into a 4L tedlar bag, which incorporated a valve, disposable mouthpiece and septum that could be pierced for sampling. Samples were collected by asking participants to exhale through the mouth into the bag until full. The valve in the bag was then closed, and samples transported immediately to the laboratory for testing.

### Analysis of Breath Samples by GC-MS

Breath samples were analysed by GC-MS for presence and quantity of pentylfuran as described for cultures above. The conditioned SPME fibre was exposed into the collection bags for 48 h and then desorbed directly in the injection port for 5 minutes.

### Results

Detection of Pentylfuran from Laboratory Cultures.

The results for testing of laboratory strains for the presence of pentylfuran are given in Table 1.

TABLE 1
Results of screening fungal and bacterial isolates by GC-MS for
presence of pentylfuran.

| Organism | Source | pentylfuran |
|---|---|---|
| A. flavus | Clinical (ear swab) | nd |
| A. flavus | Clinical (ear swab) | nd |
| A. niger | Environmental | xx |
| A. niger | Environmental | xx |
| F. oxysporum | Environmental | x |
| M. racemosus | Clinical (sinus biopsy) | x |
| C. albicans | Clinical (urine) | x |
| C. albicans | Clinical (urine) | x |
| A. fumigatus | Clinical (lung biopsy) | xx |
| A. fumigatus | Clinical (sputum) | xx |
| A. fumigatus | Clinical (sputum) | xx |
| A. fumigatus | Clinical (BAL) | xx |
| A. fumigatus | Clinical (sputum) | xx |
| A. fumigatus | Clinical (sputum) | xx |
| A. fumigatus | Clinical (sputum) | xx |
| A. fumigatus | Clinical (sputum) | xxx |
| A. fumigatus | Clinical (sputum) | xx |
| A. fumigatus | Clinical (ear swab) | xx |
| A. fumigatus | Environmental | xx |
| A. fumigatus | Environmental | xx |
| A. fumigatus | Environmental | xx |
| A. fumigatus | Environmental | xxx |
| A. fumigatus | Environmental | xxx |
| A. fumigatus | Environmental | xxx |
| A. fumigatus | Environmental | xxx |
| A. fumigatus | Environmental | xxx |
| A. fumigatus | Environmental | xx |
| A. fumigatus | Environmental | xx |
| H. influenzae | Clinical (sputum) | x |
| B. cepacia | Clinical (sputum) | x |
| Ps. aeruginosa | Clinical (sputum) | x |
| Ps. aeruginosa | Clinical (sputum) | x |
| S. aureus | Clinical (sputum) | nd |
| S. aureus | Clinical (sputum) | nd |

Source:
Spt; sputum,
BAL; bronchoalveolar lavage. pentylfuran:
nd; not detected;
x: low/trace levels,
xx; moderate levels;
xxx; high levels.

### Analysis of Breath Samples for Presence of Pentylfuran

Four litre breath samples were assayed for the presence and quantity of pentylfuran. Results are shown in table 2, along with relevant clinical and microbiological data.

TABLE 2
Demographic and microbiological data of patients enrolled in the study, and results of testing
breath by SPME/GC-MS for presence and quantity of pentylfuran. “Aspergillus colonisation” refers to
colonisation with Aspergillus fumigatus only. Organisms shown in bold are those shown to produce trace
levels of pentylfuran.. Other organisms isolated by culture of respiratory samples is shown. Organisms
shown in bold are known producers of pentylfuran.

| | | | | Underlying | S. | S. | M. | | | | 2-pentyl |
|---|---|---|---|---|---|---|---|---|---|---|---|
| | ID | Age | Sex | disease | aureus | pneumoniae | catarrhalis | [symbol] | [symbol] | [symbol] | furan (pg) |
| CF + | 12 | 20 | F | CF | | | | + | + | + | 7 |
| aspergillus | 16 | 20 | F | CF | + | | | + | | + | 3 |
| | 9 | 5 | F | CF | | | | + | | + | 7 |
| | 30 | | M | CF | | | | | + | + | 5 |
| CF, no | 10 | 7 | M | CF | + | + | | + | | | 10 |
| aspergillus | 11 | 7 | M | CF | + | + | | + | | | nd |
| | 17 | 19 | M | CF | + | | | | + | | 2 |
| | 19 | 9 | M | CF | | | | + | | | nd |
| | 20 | 7 | M | CF | | | + | + | | | nd |
| | 21 | 7 | M | CF | | + | + | + | | | 9 |
| | 22 | 8 | M | CF | | + | | + | | | nd |
| Normal | 18 | 30 | F | none | | | | | | | nd |
| controls | 13 | 25 | F | none | | | | | | | nd |
| | 14 | 33 | M | none | | | | | | | nd |
| | 15 | 31 | M | none | | | | | | | nd |
| | 24 | 35 | M | none | | | | | | | nd |
| | 25 | 36 | M | none | | | | | | | nd |
| | 26 | 36 | M | none | | | | | | | nd |
| | 27 | 44 | M | none | | | | | | | nd |
| | 28 | 37 | M | none | | | | | | | nd |
| | 29 | 57 | M | none | | | | | | | nd |

Pentylfuran was detected on the breath of all (n=4) patients colonised with A. fumigatus. Pentylfuran was also detected on breath samples of 3 of 7 patients without evidence of Aspergillus colonisation, but colonisation with other pathogens, including some shown to produce pentylfuran in modest quantities. Of the healthy individuals, one of ten subjects showed a trace level of pentylfuran on the breath.

The important message from this data set is that healthy, normal individuals appear not to produce pentylfuran (or only at baseline levels), while those with pulmonary colonisation with organisms that produce pentylfuran, do. This is the first time that the detection and quantification of a specific microbial metabolite from breath of infected/colonised individuals has been reported.

Pentylfuran is a metabolite of Aspergillus fumigatus, and possibly other fungal and bacterial pathogens. These in vitro experiments showed that low levels of pentylfuran is produced by A. flavus, as well as Pseudomonas aeruginosa and Haemophilus influenzae, both of which are common colonisers of the lungs of patients with CF.

Blood tests for Aspergillus infection are not optimal in that they are prone to sampling error, and may return false-negative results even in cases of proven disseminated infection. This may be further compounded by the administration of systemic antifungal therapy. Use of Broncho-alveloar lavage (BAL) for diagnostic purposes has had increasing support over recent years, notwithstanding that it is not an ideal procedure to be performing in a neutropenic, and often thrombocytopenic patient. However, BAL has the advantage over blood testing in that it allows direct sampling of the site of primary infection. Breath testing for metabolites of pathogens resident in the lungs may be viewed in a similar way to BAL, except that it is without the risks associated with BAL. The principle of breath testing, like BAL, is a more direct way of sampling the site of primary infection. DNA and antigen detection based assays will detect both viable and non-viable fungal cell elements. Detection of A. fumigatus metabolites from breath samples would suggest that the organism is metabolically active.

The results of this study show that:
1. Pentylfuran may be used as a biomarker of Aspergillus fumigatus, from cultures.
2. Pentylfuran can be detected from the breath of patients colonised or infected with Aspergillus fumigatus by GC-MS
3. Detection of pentylfuran from breath may form the basis of a useful diagnostic test for aspergillus infection.

### Case Report

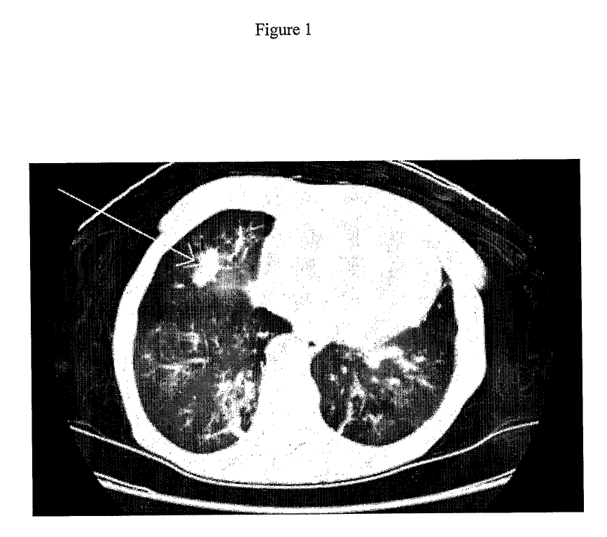

A 79 year old woman undergoing treatment with dexamethasone and cyclophosphamidefor multiple myeloma suffered an episode of febrile neutropenia (neutrophils 0.5-0.9×10⁹/L) that failed to resolve despite broad spectrum antibiotic therapy. A CT of the sinuses showed extensive disease suggestive of Aspergillus and the chest CT showed multiple foci of air space opacity with a surrounding halo of hyperdense material, suggestive of aspergillus infection (arrowed. FIG. 1). A. fumigatus was cultured from the sputum on 2 occasions. Nested PCR for A. fumigatus DNA in peripheral blood, and was negative on 6 occasions. The patient gave informed consent to participate in the study, and 3-litre breath samples were collected as described above for patients with CF. 2-pentylfuran was detected (10.1 pg) in the exhaled breath 2 days after the CT scan was performed. After treatment with voriconazole for 4 weeks, the lesions in the lungs had reduced in size, the breath test had become negative and aspergillus was no longer culturable from the sputum.

### INDUSTRIAL APPLICABILITY

The invention will be of use in the medical area, assisting in the detection of microbes and pathogens in patients. In particular, the ability to detect Aspergillus species, especially Aspergillus fumigatus will be of use in detecting and hence treatment of the infection caused by the microbe in patients.

1. A method of identifying the presence of microorganisms in the breath of an animal comprising:
providing a sample of breath from an animal; and
determining the presence or absence of an amount of pentylfuran in the sample that indicates the presence of micororganisms in the breath of said animal.

2. (canceled)

3. (canceled)

4. (canceled)

5. (canceled)

6. (canceled)

7. (canceled)

8. (canceled)

9. (canceled)

10. (canceled)

11. The method of claim 1, wherein the microorganisms are fungal pathogens.

12. The method of claim 1, wherein the microorganisms are an Aspergillus species.

13. The method of claim 1, wherein the microorganisms are Aspergillus fumigatus.

14. The method of claim 1, wherein the pentylfuran is 2-pentylfuran.

15. The method of claim 13, wherein the pentylfuran is 2-pentylfuran.

16. The method of claim 1, wherein the animal is a human.

17. The method of claim 16, wherein the microorganisms are Aspergillus fumigatus.

18. The method of claim 17, wherein the pentylfuran is 2-pentylfuran.

19. A method of detecting the presence or absence of an Aspergillus species in a sample comprising:
obtaining a gaseous sample; and
determining the presence or absence of an amount of pentylfuran in the sample that indicates the presence of an Aspergillus species in said sample.

20. The method according to claim 19, wherein the Aspergillus species is Aspergillus fumigatus.

21. The method of claim 19, wherein the pentylfuran is 2-pentylfuran.

22. The method of claim 20, wherein the pentylfuran is 2-pentylfuran.
